(19) United States
(12) Patent Application Publication
(10) Pub. No.: US 2017/0215713 A1
(43) Pub. Date: Aug. 3, 2017

(54) CAPSULE ENDOSCOPE SYSTEM AND METHOD FOR OPERATING CAPSULE ENDOSCOPE SYSTEM

(71) Applicant: OLYMPUS CORPORATION, Tokyo (JP)

(72) Inventor: Kazuhiko TAKAHASHI, Hachioji-shi, Tokyo (JP)

(73) Assignee: OLYMPUS CORPORATION, Tokyo (JP)

(21) Appl. No.: 15/489,960

(22) Filed: Apr. 18, 2017

Related U.S. Application Data
(63) Continuation of application No. PCT/JP2015/076144, filed on Sep. 15, 2015.

(30) Foreign Application Priority Data
Dec. 2, 2014 (JP) 2014-244377

(51) Int. Cl. A61B 1/045; A61B 1/00; A61B 1/04

(52) U.S. Cl. CPC A61B 1/045; A61B 1/041; A61B 1/00016; A61B 1/0002; A61B 1/00045

(57) ABSTRACT

A capsule endoscope system including: a capsule endoscope including an imaging sensor configured to capture an image of inside of a subject at a changeable imaging frame rate and generate an image signal, and an image transmitter configured to transmit a wireless signal including the image signal; and a receiving device including a first and a second antennas configured to receive the wireless signal, a receiver configured to detect a first reception intensity and a second reception intensity, a controller configured to generate a first instruction signal that changes the imaging frame rate to a first value higher than an initial value that is set in advance when the first or the second reception intensity satisfies a predetermined condition, and a transmitter configured to wirelessly transmit the first instruction signal to the capsule endoscope.

### CROSS REFERENCES TO RELATED APPLICATIONS

This application is a continuation of PCT international application Ser. No. PCT/JP2015/076144 filed on Sep. 15, 2015 which designates the United States, incorporated herein by reference, and which claims the benefit of priority from Japanese Patent Applications No. 2014-244377, filed on Dec. 2, 2014, incorporated herein by reference.

### BACKGROUND

1. Technical Field

The disclosure relates to a capsule endoscope system that acquires images in a subject by using a capsule endoscope that is introduced into the subject and captures images and to a method for operating the capsule endoscope system.

2. Related Art

In the endoscope field, a capsule endoscope that is introduced into a subject and captures images has been developed. The capsule endoscope has an imaging function and a wireless communication function in a capsule-shaped casing formed into a size that can be introduced into a digestive canal of the subject. After being swallowed into the subject, the capsule endoscope captures images while moving in the digestive canal of the subject by a peristaltic movement and the like, sequentially generates image data of images of inside of an organ of the subject (hereinafter also referred to as in-vivo images), and wirelessly transmits the image data (for example, see Japanese Translation of PCT International Application Publication No. JP-T-2010-524557). The wirelessly transmitted image data is received by a receiving device provided outside the subject and further taken into an image display device such as a workstation, and then predetermined image processing is performed on the image data. Thereby, it is possible to display the in-vivo image of the subject as a still image or a moving image.

### SUMMARY

In some embodiments, a capsule endoscope system includes: a capsule endoscope including an imaging sensor configured to capture an image of inside of a subject at a changeable imaging frame rate and generate an image signal, and an image transmitter configured to transmit a wireless signal including the image signal; and a receiving device including a first and a second antennas attached to a body surface of the subject at different positions and configured to receive the wireless signal transmitted from the capsule endoscope, a receiver configured to detect a first reception intensity which is a reception intensity of the wireless signal at the first antenna and a second reception intensity which is a reception intensity of the wireless signal at the second antenna, a controller configured to generate a first instruction signal that changes the imaging frame rate to a first value higher than an initial value that is set in advance when the first or the second reception intensity satisfies a predetermined condition, and a transmitter configured to wirelessly transmit the first instruction signal to the capsule endoscope.

In some embodiments, provided is a method for operating a capsule endoscope system including: a capsule endoscope configured to capture an image of inside of a subject at a changeable imaging frame rate, generate an image signal, and transmit a wireless signal including the image signal; and a receiving device including a first and a second antennas attached to a body surface of the subject at different positions and configured to receive the wireless signal transmitted from the capsule endoscope. The method includes: detecting, by the receiving device, a first reception intensity which is a reception intensity of the wireless signal at the first antenna and a second reception intensity which is a reception intensity of the wireless signal at the second antenna, generating, by the receiving device, a first instruction signal that changes the imaging frame rate to a first value higher than an initial value that is set in advance when the first or the second reception intensity satisfies a predetermined condition, and wirelessly transmitting, by the receiving device, the first instruction signal to the capsule endoscope.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a configuration example of a capsule endoscope system according to a first embodiment of the disclosure;

FIG. 2 is a schematic diagram illustrating an example of an internal structure of a capsule endoscope illustrated in FIG. 1;

FIG. 3 is a flowchart illustrating an operation of the capsule endoscope illustrated in FIG. 2;

FIG. 4 is a flowchart illustrating an operation of a receiving device illustrated in FIG. 1;

FIG. 5 is a graph illustrating a relationship between temporal variations of reception intensities at two receiving antennas and a timing of changing an imaging frame rate;

FIG. 6 is a schematic diagram illustrating an image sequence based on a series of image signals stored in a memory illustrated in FIG. 1;

FIG. 7 is a schematic diagram illustrating an image sequence based on a series of image signals acquired in a first modified example of the first embodiment of the disclosure;

FIG. 8 is a flowchart illustrating an operation of a receiving device included in a capsule endoscope system according to a second embodiment of the disclosure; and

FIG. 9 is a graph illustrating a relationship between temporal variations of reception intensities at two receiving antennas and a timing of changing an imaging frame rate.

### DETAILED DESCRIPTION

Hereinafter, a capsule endoscope system and a method for operating the capsule endoscope system according to embodiments of the disclosure will be described with reference the drawings. In the description below, each drawing merely schematically illustrates shapes, sizes, and positional relationships in a degree such that contents of the disclosure can be understood. Therefore, the disclosure is not limited to the sizes, the shapes, and the positional relationships illustrated in each drawing. In the description of the drawings, the same components are denoted by the same reference numerals.

### First Embodiment

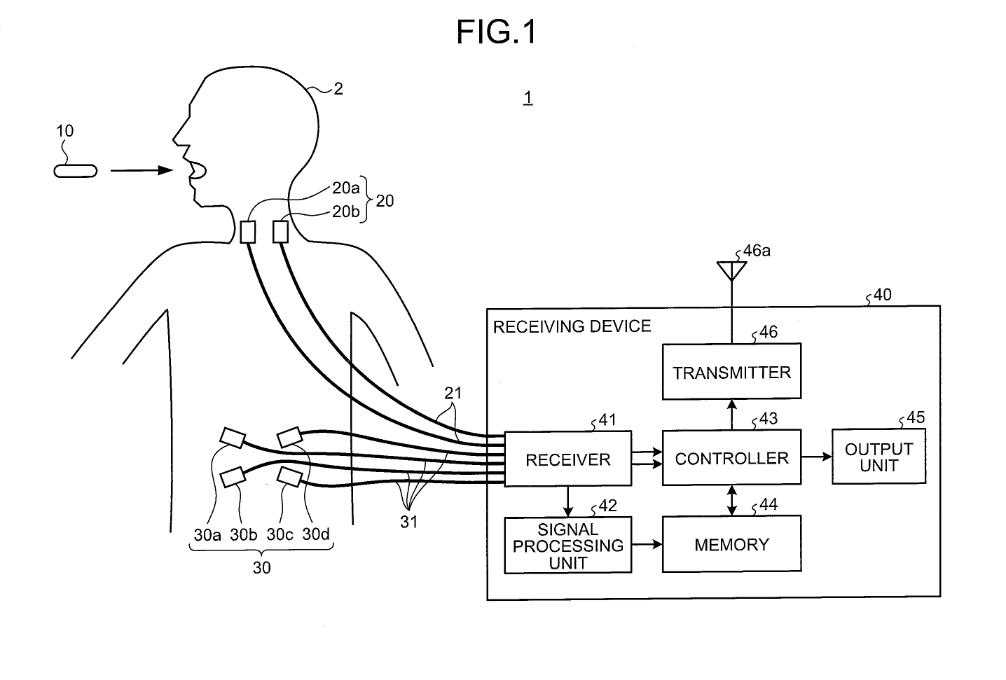

FIG. 1 is a schematic diagram illustrating a configuration example of a capsule endoscope system according to a first embodiment of the disclosure. A capsule endoscope system 1 illustrated in FIG. 1 includes a capsule endoscope 10 that is introduced into a lumen (digestive canal) of a subject 2 such as a patient, captures images, and transmits a wireless signal including an image signal, two receiving antenna groups 20 and 30 that receive the wireless signal transmitted from the capsule endoscope 10, and a receiving device 40 that takes in an electrical signal corresponding to the wireless signal received by the receiving antenna groups 20 and 30 and acquires an image signal by performing predetermined processing on the electrical signal.

The capsule endoscope 10 is introduced into the subject by, for example, oral ingestion, and thereafter moves in a lumen (digestive canal) of the subject, and is finally discharged to the outside of the subject. During that time, the capsule endoscope 10 captures images of the inside of an organ of the subject, generates image signals, and sequentially wirelessly transmits the image signals to the outside of the subject 2.

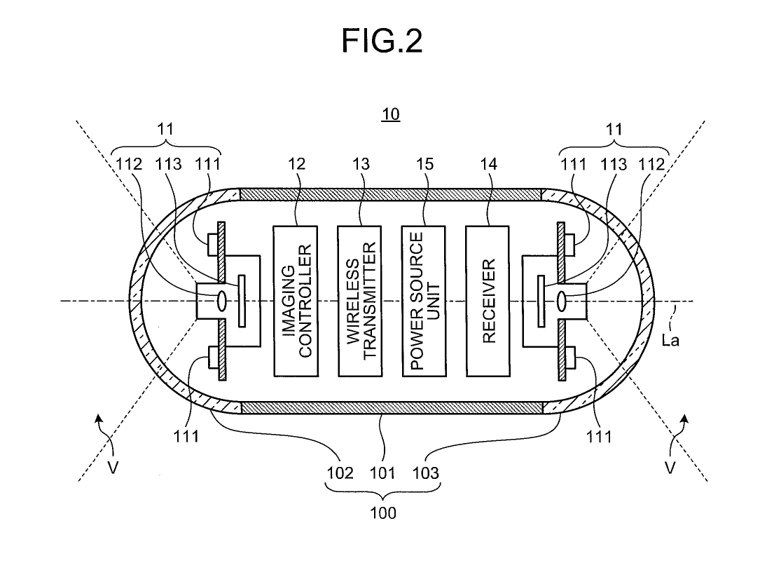

FIG. 2 is a schematic diagram illustrating an example of an internal structure of the capsule endoscope 10. As illustrated in FIG. 2, the capsule endoscope 10 includes a capsule-shaped casing 100 that is an outer casing formed into a size that can be easily introduced into the inside of an organ of the subject 2, two imaging units 11 (imaging sensors) that capture images of the subject in directions different from each other, an imaging controller 12 that processes a signal inputted from each imaging unit 11 and controls each component of the capsule endoscope 10, a wireless transmitter 13 that wirelessly transmits the signal processed by the imaging controller 12 to the outside of the capsule endoscope 10, a receiver 14 that receives an instruction signal and the like wirelessly transmitted from the outside, and a power source unit 15 that supplies power to each component of the capsule endoscope 10.

The capsule-shaped casing 100 includes a tubular casing 101 and dome-shaped casings 102 and 103 and is formed by closing open ends of both sides of the tubular casing 101 with the dome-shaped casings 102 and 103. The tubular casing 101 is a colored casing that is substantially opaque to visible light. On the other hand, the dome-shaped casings 102 and 103 are dome-shaped optical members that are transparent to light of a predetermined wavelength band such as visible light. The capsule-shaped casing 100 internally includes the imaging unit 11, the imaging controller 12, the wireless transmitter 13, the receiver 14, and the power source unit 15 in a liquid-tight manner.

Each imaging unit 11 has an illumination unit 111 which is formed of LED (Light Emitting Diode), LD (Laser Diode), or the like and emits illumination light such as white light, an optical system 112 such as a condenser lens, and an imaging element 113 formed of a CMOS image sensor, a CCD, or the like. The illumination unit 111 emits illumination light to the subject in a visual field V of each imaging element 113 through the dome-shaped casing 102 or 103. The optical system 112 condenses reflection light from the subject in the visual field V and forms an image on an imaging surface of the imaging element 113. The imaging element 113 converts the reflection light (optical signal) from the subject in the visual field V, which forms an image on the imaging surface, into an electrical signal and outputs the electrical signal as an image signal.

The two imaging units 11 are arranged so that optical axes of the optical systems 112 of the two imaging units 11 are substantially in parallel with or substantially coincident with a long axis La which is a center axis in a longer direction of the capsule-shaped casing 100 and that the visual fields V of the two imaging units 11 face directions opposite to each other. In other words, the two imaging units 11 are mounted so that the imaging surface of each imaging element 113 is perpendicular to the long axis La.

The first embodiment employs a compound eye type capsule endoscope in which the two imaging units 11 captures images, respectively, in both directions (front and rear directions) of the long axis La of the capsule endoscope 10. However, a single eye type capsule endoscope may be employed in which only one imaging unit 11 is provided and images are captured in one direction of the long axis La.

The imaging controller 12 controls an imaging operation of the imaging unit 11 and controls an operation of each component of the capsule endoscope 10, and further controls input and output of signals between these components. Specifically, the imaging controller 12 sets an imaging frame rate of the imaging unit 11, causes the illumination unit 111 to emit light in synchronization with the set imaging frame rate, causes the imaging element 113 to capture images of the subject in the visual field V illuminated by the illumination unit 111, and further performs predetermined signal processing on the image signal outputted from the imaging element 113.

The wireless transmitter 13 includes an antenna for transmitting a wireless signal. The wireless transmitter 13 acquires the image signal on which the signal processing is performed by the imaging controller 12, generates a wireless signal by performing modulation processing and the like on the image signal, and transmits the wireless signal.

The receiver 14 receives various instruction signals wirelessly transmitted from the receiving device 40, performs demodulation processing and the like on the instruction signals, and then outputs the instruction signals to the imaging controller 12.

The power source unit 15 is a power storage unit such as a button-type battery or a capacitor and has a switch unit such as a magnetic switch or an optical switch. When the power source unit 15 is configured to have a magnetic switch, the power source unit 15 switches between ON and OFF states of a power source by a magnetic field applied from outside. When the power source is in an ON state, the power source unit 15 supplies power of the power storage unit to each component (the imaging unit 11, the imaging controller 12, the wireless transmitter 13, and the receiver 14) of the capsule endoscope 10. When the power source is in an OFF state, the power source unit 15 stops power supply to each component of the capsule endoscope 10.

Referring to FIG. 1 again, the receiving antenna group 20 includes at least one (in FIG. 1, two) receiving antennas 20a and 20b and at least one (in FIG. 1, two) cables 21 that respectively connect the receiving antennas 20a and 20b to the receiving device 40. Each of the receiving antennas 20a and 20b is a sheet-shaped loop antenna or dipole antenna, which is formed by printing an antenna circuit on a sheet-shaped flexible substrate. Each of the receiving antennas 20a and 20b is attached to the body surface (or the surface of clothing) of the subject at a predetermined position near the esophagus by using, for example, an adhesive seal. In FIG. 1, the receiving antennas 20a and 20b are respectively attached to the left and right sides of the neck of the subject 2. Hereinafter, the receiving antennas 20a and 20b are also referred to as esophagus antennas 20a and 20b.

The receiving antenna group 30 includes a plurality of (four in FIG. 1) receiving antennas 30a to 30d and a plurality of (four in FIG. 1) cables 31 that respectively connect the receiving antennas 30a to 30d to the receiving device 40. In the same manner as the receiving antennas 20a and 20b, each of the receiving antennas 30a to 30d is a sheet-shaped loop antenna or dipole antenna, which is formed by printing an antenna circuit on a sheet-shaped flexible substrate. Each of the receiving antennas 30a to 30d is attached to the body surface (or the surface of clothing) of the subject at a predetermined position near the abdomen by using, for example, an adhesive seal. Hereinafter, the receiving antennas 30a to 30d are also referred to as abdomen antennas 30a to 30d.

These receiving antennas 20a, 20b, and 30a to 30d are connected to a predetermined connector of a receiver 41 included in the receiving device 40, which is described below, through the cables 21 and 31. The structures (i.e. sizes and circuit configurations) of the receiving antennas 20a and 20b may be the same as those of the receiving antennas 30a to 30d or may be different from those of the receiving antennas 30a to 30d. However, it is preferable to attach labels, each of which contains an identifiable sign or the like, to the receiving antennas 20a, 20b, and 30a to 30d, respectively, so as not to mistake the positions where the receiving antennas 20a, 20b, and 30a to 30d are attached to the subject 2. Further, the lengths of the cables 21 and 31 may be changed according to the positions on the body surface of the subject 2, at which the receiving antennas 20a, 20b, and 30a to 30d are attached. For example, as illustrated in FIG. 1, when the receiving device 40 is arranged near the lower back of the subject 2, it is preferable that the cables 21 to be connected to the esophagus antennas 20a and 20b are longer than the cables 31 to be connected to the abdomen antennas 30a to 30d.

The receiving device 40 includes: the receiver 41 that acquires an electrical signal corresponding to a wireless signal, which is transmitted from the capsule endoscope 10, through the receiving antenna groups 20 and 30; a signal processing unit 42 that extracts an image signal by performing predetermined signal processing on the electrical signal; a controller 43 that integrally controls each component of the receiving device 40 and generates an instruction signal to the capsule endoscope 10; a memory 44 that stores image signals, which are extracted by the signal processing unit 42, in chronological order; an output unit 45 that outputs the image signal stored in the memory 44 to an external device such as an image display device; and a transmitter 46 that wirelessly transmits the instruction signal generated by the controller 43.

The receiver 41 has a connector to which the cables 21 and 31 are detachably connected. The receiver 41 detects reception intensities of the wireless signal at the receiving antennas 20a, 20b, and 30a to 30d based on signals inputted through the cables 21 and 31 and the connector and outputs an electrical signal corresponding to a wireless signal received by a receiving antenna whose reception intensity is the strongest to the signal processing unit 42. Further, the receiver 41 outputs a signal representing reception intensities at two receiving antennas, which are respectively selected from the receiving antenna groups 20 and 30, to the controller 43.

The signal processing unit 42 extracts an image signal by performing predetermined signal processing such as demodulation processing on the electrical signal outputted from the receiver 41 and stores the image signal and related information (time information and the like) in the memory 44 in chronological order.

The controller 43 compares the reception intensities at the two receiving antennas, which are outputted from the receiver 41, and generates an instruction signal to change an imaging frame rate in the capsule endoscope 10 based on a result of the comparison. Then, the controller 43 outputs the instruction signal from the transmitter 46.

The output unit 45 is an interface that connects the receiving device 40 to an external device such as an image display device. The output unit 45 outputs the image signal and related information stored in the memory 44 to the external device such as an image display device.

The transmitter 46 has a transmitting antenna 46a. The transmitter 46 generates a wireless signal by performing modulation processing and the like on the instruction signal generated by the controller 43 and transmits the wireless signal to the capsule endoscope 10 through the transmitting antenna 46a.

The receiving device 40 as described above is carried by the subject 2 while an examination using the capsule endoscope 10 is being performed. For example, it is preferable to attach the receiving device 40 around the waist of the subject 2 by using a belt or the like.

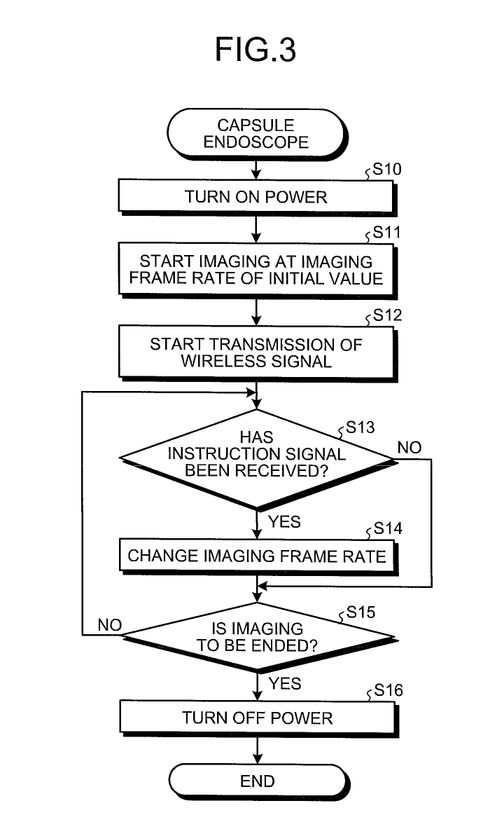

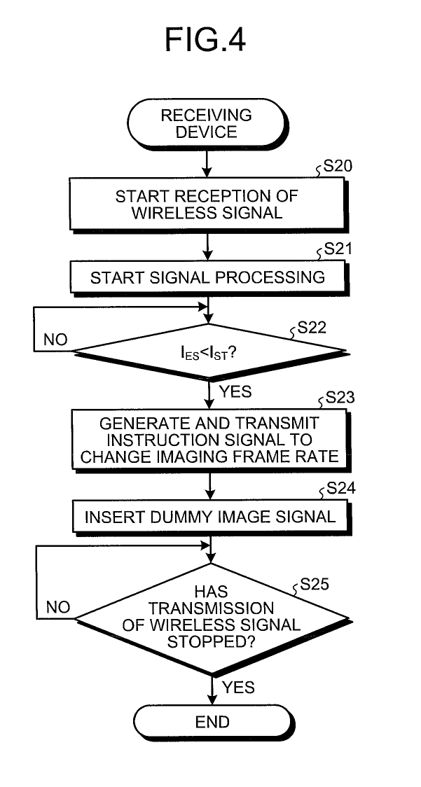

Next, an operation of the capsule endoscope system 1 will be described. FIG. 3 is a flowchart illustrating an operation of the capsule endoscope 10. FIG. 4 is a flowchart illustrating an operation of the receiving device 40.

First, in step S10 illustrated in FIG. 3, a user (i.e. a medical worker in charge of examination) turns on power of the capsule endoscope 10 by using a magnetic switch or the like. Thereby, power supply from the power source unit 15 is started to each functional unit included in the capsule endoscope 10.

In step S11, the imaging unit 11 starts imaging at an imaging frame rate that is set in advance as an initial value. Specifically, the illumination unit 111 starts light emission in accordance with the set imaging frame rate, and the imaging element 113 starts imaging at the set imaging frame rate, generates an image signal, and outputs the image signal.

In the first embodiment, a high-speed imaging frame rate (for example, 20 to 60 fps) suitable for observing esophagus is set as the initial value of the imaging frame rate. This is because the capsule endoscope 10 passes through the esophagus in a short time, so that a high-speed imaging frame rate is required to sufficiently observe the esophagus.

In the subsequent step S12, the wireless transmitter 13 starts an operation to generate and transmit a wireless signal by performing modulation processing or the like on the image signal outputted from the imaging unit 11.

In step S20 illustrated in FIG. 4, the receiving device 40 starts reception of the wireless signal transmitted from the capsule endoscope 10 through the receiving antenna groups 20 and 30. At this time, the receiver 41 detects reception intensities of the wireless signal at the receiving antennas 20a, 20b, and 30a to 30d and outputs an electrical signal corresponding to a wireless signal received by a receiving antenna whose reception intensity is the strongest to the signal processing unit 42. Further, the receiver 41 outputs a signal representing reception intensities at two receiving antennas, which are respectively selected from the receiving antenna groups 20 and 30, to the controller 43. In the first embodiment, it is assumed that the esophagus antenna 20b of the receiving antenna group 20 and the abdomen antenna 30d of the receiving antenna group 30 are selected as the receiving antennas from which the reception intensities are outputted.

In the subsequent step S21, the signal processing unit 42 starts signal processing to extract an image signal by performing signal processing such as demodulation processing and the like on the electrical signal outputted from the receiver 41. The extracted image signal is sequentially stored in the memory 44.

At this stage, the user confirms that the capsule endoscope 10 starts an operation and causes the subject 2 to swallow the capsule endoscope 10. Specifically, the user checks whether the illumination unit 111 of the capsule endoscope 10 periodically emits light and whether the receiving device 40 receives the wireless signal transmitted from the capsule endoscope 10.

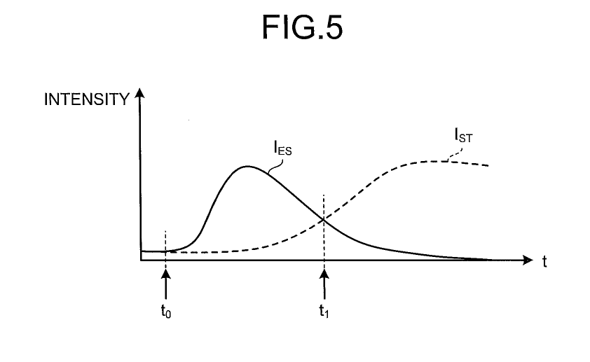

In step S22, the controller 43 starts comparison determination of reception intensities based on the signals representing the reception intensities at two receiving antennas which is outputted from the receiver 41. Here, FIG. 5 is a graph illustrating a relationship between temporal variations of reception intensities at two receiving antennas and a timing of changing the imaging frame rate. The solid line illustrated in FIG. 5 illustrates a temporal change of a reception intensity I_{ES} at the esophagus antenna 20b and the dashed line illustrated in FIG. 5 illustrates a temporal change of a reception intensity I_{ST} at the abdomen antenna 30d.

At a time point when the subject 2 puts the capsule endoscope 10 into his or her mouth (t=t₀), the capsule endoscope 10 is located closer to the esophagus than to the stomach of the subject 2. Therefore, the reception intensity I_{ES} at the esophagus antenna 20b is stronger than the reception intensity I_{ST} at the abdomen antenna 30d. Thereafter, when the subject 2 swallows the capsule endoscope 10, the capsule endoscope 10 passes through the esophagus and reaches the stomach. Specifically, the capsule endoscope 10 rapidly approaches the esophagus antenna 20b and then goes away from the esophagus antenna 20b. On the other hand, the capsule endoscope 10 gradually approaches the abdomen antenna 30d. Therefore, the reception intensity I_{ES} at the esophagus antenna 20b rapidly rises after time t=t₀, reaches a peak, and then falls. On the other hand, the reception intensity I_{ST} at the abdomen antenna 30d gradually rises.

As illustrated in FIG. 5, a timing (t=t₁) when the strength relation of the reception intensities between the reception intensity I_{ES} and the reception intensity I_{ST} is reversed is a timing when a distance from the capsule endoscope 10 to the esophagus antenna 20b and a distance from the capsule endoscope 10 to the abdomen antenna 30d are reversed, that is to say, a timing when the capsule endoscope 10 becomes relatively closer to the abdomen antenna 30d. After the timing, the wireless signal transmitted by the capsule endoscope 10 is mainly received by the abdomen antennas 30a to 30d, so that it can be assumed that the timing (t=t₁) is a timing when the capsule endoscope 10 enters the stomach.

Therefore, the controller 43 determines whether or not the reception intensity I_{ST} at the abdomen antenna 30d is greater than the reception intensity I_{ES} at the esophagus antenna 20b. When the reception intensity I_{ST} at the abdomen antenna 30d is smaller than or equal to the reception intensity I_{ES} at the esophagus antenna 20b (step S22: No), the controller 43 continuously performs the comparison determination on the reception intensities I_{ES} and I_{ST}.

On the other hand, when the reception intensity I_{ST} at the abdomen antenna 30d is greater than the reception intensity I_{ES} at the esophagus antenna 20b (step S22: Yes), the controller 43 assumes that the capsule endoscope 10 passes through the esophagus and enters the stomach, generates an instruction signal to change the imaging frame rate in the capsule endoscope 10, and transmits the instruction signal through the transmitter 46 (step S23). Specifically, the controller 43 generates an instruction signal to change the imaging frame rate to a lower value (for example, about 2 fps). This is because the capsule endoscope 10 stays inside the stomach for a relatively long time, so that, when imaging the inside of the stomach, a high-speed imaging frame rate used when imaging the esophagus is not required. After generating and transmitting the instruction signal to change the imaging frame rate, the controller 43 ends the comparison determination on the reception intensities I_{ES} and I_{ST} (see step S22).

In the subsequent step S24, the controller 43 generates a dummy image signal and inserts and stores the dummy image signal into a sequence of image signals stored in chronological order in the memory 44. A timing of inserting the dummy image signal is a timing of transmitting the instruction signal to change the imaging frame rate or a timing a predetermined time after the timing of transmitting the instruction signal.

The content of the dummy image signal is not particularly limited as long as the user can identify an image based on the dummy image signal from images of the inside of the subject 2. For example, an image signal representing an image of white paper may be inserted as the dummy image signal. The dummy image signal that forms a dummy image can be held in the controller 43 in advance.

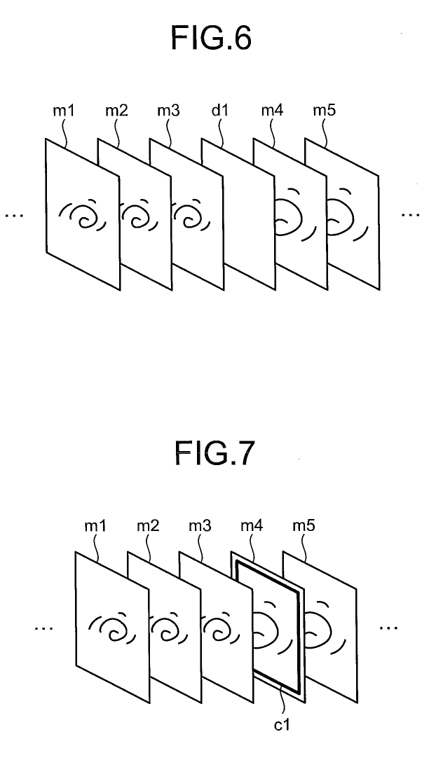

FIG. 6 is a schematic diagram illustrating an image sequence based on a series of image signals stored in the memory 44. In FIG. 6, as an example, a dummy image dl of white paper is inserted into a sequence of images m1 to m5 in chronological order based on image signals generated by the capsule endoscope 10. By inserting such a dummy image dl into the image sequence, the user can easily identify the images m1 to m3 captured before the imaging frame rate is changed and the images m4 and m5 captured after the imaging frame rate is changed.

In step S13 illustrated in FIG. 3, the imaging controller 12 of the capsule endoscope 10 determines whether or not the receiver 14 has received the instruction signal from the receiving device 40. When the receiver 14 has not received the instruction signal (step S13: No), the operation of the capsule endoscope 10 proceeds to step S15 described below. In this case, the imaging unit 11 continuously performs imaging at the imaging frame rate that has been used.

On the other hand, when the receiver 14 has received the instruction signal (step S13: Yes), the imaging controller 12 performs control to change the imaging frame rate in the imaging unit 11 (step S14). For example, when an instruction signal to change the imaging frame rate to a lower value is transmitted from the receiving device 40, the imaging controller 12 lowers the imaging frame rate in the imaging unit 11 to an instructed imaging frame rate. Thereafter, the imaging unit 11 performs imaging at the changed imaging frame rate.

In the subsequent step S15, the imaging controller 12 determines whether or not to end the imaging. Specifically, the imaging controller 12 determines to end the imaging when a predetermined time has elapsed since the capsule endoscope 10 was started or when a battery residual capacity becomes lower than a predetermined value.

When the imaging is not to be ended (step S15: No), the operation of the capsule endoscope 10 returns to step S13. On the other hand, when the imaging is to be ended (step S15: Yes), the imaging controller 12 turns off power supply from the power source unit 15 to each functional unit (step S16). Thereby, the capsule endoscope 10 ends the operation.

In step S25 illustrated in FIG. 4, the receiving device 40 determines whether or not the transmission of the wireless signal from the capsule endoscope 10 has stopped. When the transmission of the wireless signal continues (step S25: No), the receiving device 40 continues reception of the wireless signal and signal processing. On the other hand, when the transmission of the wireless signal has stopped (step S25: Yes), the receiving device 40 ends operation.

The image signals that are accumulated in the memory 44 of the receiving device 40 in this way are transferred to an image display device or the like through the output unit 45 when the receiving device 40 is connected to an external device such as the image display device or the like. The image display device or the like displays a series of images (see FIG. 6) based on the image signals as a moving image or a list of still images.

As described above, according to the first embodiment of the disclosure, the receiving device 40 generates an instruction signal to change the imaging frame rate in the capsule endoscope 10 based on a comparison result between the reception intensity at the receiving antenna 20b attached to the body surface near the esophagus of the subject 2 and the reception intensity at the receiving antenna 30d attached to the body surface near the abdomen and wirelessly transmits the instruction signal, and the capsule endoscope 10 changes the imaging frame rate according to the instruction signal. Thus, it is possible to appropriately control the imaging frame rate according to an observed region without complicating the configuration of the capsule endoscope 10.

Further, according to the first embodiment described above, when the instruction signal to change the imaging frame rate is transmitted from the receiving device 40, a dummy image signal is inserted into the sequence of the image signals stored in chronological order. Therefore, when the user observes the image sequence based on the image signals, the user can easily identify an image group captured before the imaging frame rate is changed and an image group captured after the imaging frame rate is changed.

In the first embodiment described above, there may be a case in which it is determined that the capsule endoscope 10 has moved from the esophagus to the stomach and thereafter the reception intensity of the esophagus antenna 20b becomes again stronger than that of the abdomen antenna 30d, that is to say, a phenomenon may occur in which it seems that the capsule endoscope 10 flows back to the esophagus. However, even when such a phenomenon occurs, the phenomenon can be handled as an error and it is not particularly necessary to further control the imaging frame rate. This is because the capsule endoscope 10 has passed through the esophagus where the fastest imaging frame rate is required. Therefore, in the entire examination using the capsule endoscope 10, the control to change the imaging frame rate from a high value suitable for observing the esophagus to a low value suitable for observing the stomach and organs located beyond the stomach only has to be performed once.

### First Modified Example

Next, a first modified example of the first embodiment of the disclosure will be described.

In the first embodiment described above, when the receiving device 40 transmits the instruction signal to change the imaging frame rate, the receiving device 40 inserts a dummy image signal into a sequence of image signals stored in chronological order. However, instead of inserting the dummy image signal, information indicating that the imaging frame rate is changed may be added to the image signals to be stored in the memory 44. The information indicating that the imaging frame rate is changed may be, for example, textual information added to an edge portion of an image or graphic information such as a sign and a frame.

FIG. 7 is a schematic diagram illustrating an image sequence based on a series of image signals acquired in the first modified example. In FIG. 7, among images m1 to m5 in chronological order based on a series of image signals, a frame c1 is added, as information indicating that the imaging frame rate is changed, to the image m4 based on an image signal that is generated immediately after the imaging frame rate is changed. Therefore, by seeing the image m4 to which the frame c1 is added, the user can easily identify the images m1 to m3 captured before the imaging frame rate is changed and the images m4 and m5 captured after the imaging frame rate is changed.

### Second Modified Example

Next, a second modified example of the first embodiment of the disclosure will be described.

The receiving device 40 may be further provided with a notification unit for notifying the user of an operation and the like of each component. For example, an LED lamp that turns on under control of the controller 43 is provided as the notification unit, and the LED lamp may be turned on when the receiving device 40 starts reception of the wireless signal transmitted from the capsule endoscope 10. Thereby, the user can confirm that the capsule endoscope 10 normally operates and can instruct the subject 2 to swallow the capsule endoscope 10.

Alternatively, an LED lamp that turns on under control of the controller 43 is provided as the notification unit, and the LED lamp may be turned on when the instruction signal to change the imaging frame rate in the capsule endoscope 10 is transmitted from the receiving device 40. Thereby, the user can know that the capsule endoscope 10 reaches the stomach and thereafter the user can perform an operation such as removing the esophagus antennas 20a and 20b, which will not be used, from the subject 2 and the receiving device 40.

As the notification unit, besides the LED lamp described above, it is possible to provide a display unit that displays a text message, a speaker that outputs voice and alarm sound, and the like.

### Second Embodiment

Next, a second embodiment of the disclosure will be described.

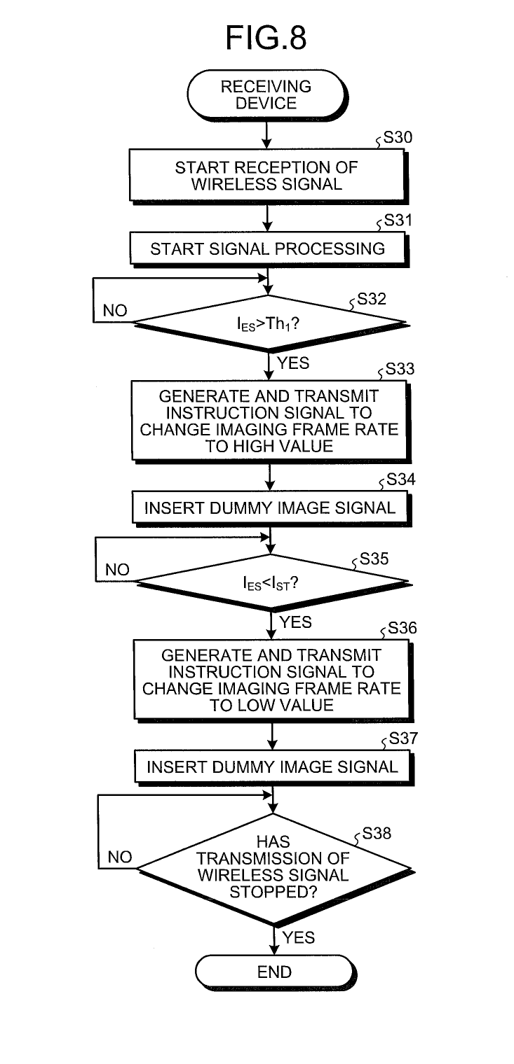

A configuration of a capsule endoscope system according to the second embodiment is the same as that of the first embodiment (see FIGS. 1 and 2), and in the second embodiment, an operation of the receiving device 40 illustrated in FIG. 1 is different from that of the first embodiment. FIG. 8 is a flowchart illustrating an operation of the receiving device 40 in the second embodiment.

An operation of a capsule endoscope 10 in the second embodiment is the same as that illustrated in FIG. 3. However, a low value (for example, 2 fps) is set as the initial value of the imaging frame rate (see step S11 in FIG. 3). This is to suppress useless power consumption required for the imaging operation and the transmission operation of the wireless signal because imaging is not substantively performed in the subject 2 from when the power of the capsule endoscope 10 is turned on to when a subject 2 swallows the capsule endoscope 10.

Steps S30 and S31 illustrated in FIG. 8 correspond to steps S20 and S21 illustrated in FIG. 4 (see the first embodiment). At this stage, the user confirms that the capsule endoscope 10 starts an operation and instructs the subject 2 to swallow the capsule endoscope 10.

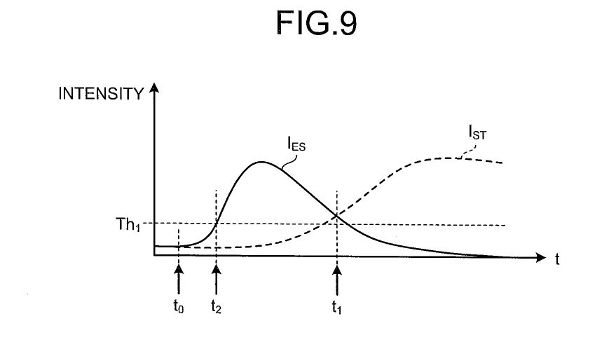

In step S32 subsequent to step S31, a controller 43 starts determination of the reception intensity based on a signal representing the reception intensity at an esophagus antenna 20b of signals representing the reception intensities at two receiving antennas 20b and 30d that are selected in advance. Here, FIG. 9 is a graph illustrating a relationship between temporal variations of reception intensities of two receiving antennas and a timing of changing the imaging frame rate. The solid line illustrated in FIG. 9 illustrates a temporal change of a reception intensity I_{ES} at the esophagus antenna 20b and the dashed line illustrated in FIG. 9 illustrates a temporal change of a reception intensity I_{ST} at the abdomen antenna 30d.

The controller 43 determines whether or not the reception intensity I_{ES} at the esophagus antenna 20b is greater than a threshold value Th₁. The threshold value Th₁ is set in advance based on statistics of the reception intensity at the esophagus antenna 20b of when the capsule endoscope 10 passes near the throat of the subject 2.

When the reception intensity I_{ES} at the esophagus antenna 20b is smaller than or equal to the threshold value Th₁ (step S32: No), the controller 43 continuously performs the determination of the reception intensity I_{ES}.

On the other hand, when the reception intensity I_{ES} at the esophagus antenna 20b is smaller than or equal to the threshold value Th₁ (step S32: Yes), the controller 43 assumes that the subject 2 swallows the capsule endoscope 10 (in other words, the capsule endoscope 10 passes through the throat of the subject 2) and the capsule endoscope 10 enters the esophagus, and the controller 43 generates an instruction signal to change the imaging frame rate in the capsule endoscope 10 to a high value (for example, 20 to 60 fps) suitable for observing the esophagus and transmits the instruction signal through the transmitter 46 (step S33).

Accordingly, the capsule endoscope 10 changes the imaging frame rate in the imaging unit 11 to a high value (see step S14 in FIG. 3) according to the received instruction signal.

In step S34, the controller 43 generates a dummy image signal, inserts the dummy image signal into a sequence of image signals stored in chronological order, and causes the memory 44 to store the dummy image signal. The timing of inserting the dummy image signal is a timing of transmitting the instruction signal to change the imaging frame rate or a timing a predetermined time after the timing of transmitting the instruction signal. The content of the dummy image signal may be an image signal representing an image of white paper in the same manner as in the first embodiment. Alternatively, in the same manner as in the first modified example of the first embodiment, instead of inserting the dummy image signal, information indicating that the imaging frame rate is changed may be added to the image signals.

In the subsequent step S35, the controller 43 determines whether or not the reception intensity I_{ST} at the abdomen antenna 30d is greater than the reception intensity I_{ES} at the esophagus antenna 20b. When the reception intensity I_{ST} at the abdomen antenna 30d is smaller than or equal to the reception intensity I_{ES} at the esophagus antenna 20b (step S35: No), the controller 43 continuously performs the determination on the reception intensities I_{ES} and I_{ST}.

On the other hand, when the reception intensity I_{ST} at the abdomen antenna 30d is greater than the reception intensity I_{ES} at the esophagus antenna 20b (step S35: Yes), the controller 43 assumes that the capsule endoscope 10 passes through the esophagus and enters the stomach, generates an instruction signal to change the imaging frame rate in the capsule endoscope 10 to a lower value (for example, 2 fps), and transmits the instruction signal through the transmitter 46 (step S36).

Accordingly, the capsule endoscope 10 changes the imaging frame rate in the imaging unit 11 to a low value (see step S14 in FIG. 3) according to the received instruction signal.

In step S37, the controller 43 generates a dummy image signal, inserts the dummy image signal into a sequence of image signals stored in chronological order, and causes the memory 44 to store the dummy image signal in the same manner as in step S34.

After transmitting the instruction signal to change the imaging frame rate to a lower value, the controller 43 ends the determination on the reception intensities I_{ES} and I_{ST} (see steps S32 and S35). At this time, the user may remove the esophagus antennas 20a and 20b from the subject 2 and the receiving device 40.

In the subsequent step S38, the receiving device 40 determines whether or not the transmission of the wireless signal from the capsule endoscope 10 has stopped. When the transmission of the wireless signal continues (step S38: No), the receiving device 40 continues reception of the wireless signal and signal processing. On the other hand, when the transmission of the wireless signal has stopped (step S38: Yes), the receiving device 40 ends operation.

As described above, according to the second embodiment of the disclosure, the timing (t=t₂) when the subject 2 swallows the capsule endoscope 10 is determined based on the reception intensity at the esophagus antenna 20b, and the imaging frame rate is changed from a low value (initial value) to a high value at the timing. Thus, it is possible to suppress power consumption during a period from when the power of the capsule endoscope 10 is turned on to when the subject 2 swallows the capsule endoscope 10. Thereafter, the timing (t=t₁) when the capsule endoscope 10 moves from the esophagus to the stomach is determined based on the reception intensities at the esophagus antenna 20b and the abdomen antenna 30d, and the imaging frame rate is changed to a low value at the timing. Thus, it is possible to perform the imaging at an appropriate imaging frame rate suitable for an observed region in the subject 2.

In the second embodiment described above, the timing when the subject 2 swallows the capsule endoscope 10 is determined based on the reception intensity at the esophagus antenna 20b. However, a timing when the subject 2 puts the capsule endoscope 10 into his or her mouth may be determined based on the reception intensity at the abdomen antenna 30d and, at the timing, an instruction signal to change the imaging frame rate in the capsule endoscope 10 to a high value may be generated and transmitted. In this case, as a threshold value for the determination, a value smaller than the threshold value Th₁ may be set.

According to the some embodiments, the receiving device compares the reception intensities at the two antennas that receive the wireless signal transmitted from the capsule endoscope, and the receiving device transmits the instruction signal that changes the imaging frame rate in the capsule endoscope based on a result of the comparison. The capsule endoscope changes the imaging frame rate according to the instruction signal, so that it is possible to appropriately control the imaging frame rate according to an observed region, without complicating the configuration of the capsule endoscope.

The first and the second embodiments and the modified examples described above are merely examples for implementing the present invention, and the present invention is not limited to these embodiments and modified examples. The disclosure can form various inventions by appropriately combining a plurality of components disclosed in the first and the second embodiments and the modified examples. From the above description, it is obvious that the disclosure can be variously modified according to specifications and the like, and further, other various embodiments are possible within the scope of the present invention.

What is claimed is:

1. A capsule endoscope system comprising:
a capsule endoscope including
an imaging sensor configured to capture an image of inside of a subject at a changeable imaging frame rate and generate an image signal, and
an image transmitter configured to transmit a wireless signal including the image signal; and
a receiving device including
a first and a second antennas attached to a body surface of the subject at different positions and configured to receive the wireless signal transmitted from the capsule endoscope,
a receiver configured to detect a first reception intensity which is a reception intensity of the wireless signal at the first antenna and a second reception intensity which is a reception intensity of the wireless signal at the second antenna,
a controller configured to generate a first instruction signal that changes the imaging frame rate to a first value higher than an initial value that is set in advance when the first or the second reception intensity satisfies a predetermined condition, and
a transmitter configured to wirelessly transmit the first instruction signal to the capsule endoscope.

2. The capsule endoscope system according to claim 1, wherein the controller is configured to generate the first instruction signal that changes the imaging frame rate to the first value higher than the initial value when the first reception intensity becomes stronger than a threshold value.

3. The capsule endoscope system according to claim 2, wherein
the controller is configured to generate a second instruction signal that changes the imaging frame rate to a second value lower than the first value when a strength relation of reception intensities between the first reception intensity and the second reception intensity is reversed, and
the transmitter is configured to transmit the second instruction signal.

4. The capsule endoscope system according to claim 3, wherein
the first antenna is attached to the body surface at a position where the capsule endoscope passes through the subject at a first passing speed,
the second antenna is attached to the body surface at a position where the capsule endoscope passes through the subject at a second passing speed slower than the first passing speed, and
the controller is configured to generate the first instruction signal when the first reception intensity becomes stronger than the threshold value, and generate the second instruction signal when a state in which the first reception intensity is stronger than the second reception intensity is changed to a state in which the second reception intensity is stronger than the first reception intensity.

5. The capsule endoscope system according to claim 3, wherein
the first antenna is attached to the body surface at the subject closer to an esophagus of the subject as compared with the second antenna,
the second antenna is attached to the body surface at the subject closer to a stomach of the subject as compared with the first antenna, and
the controller is configured to generate the first instruction signal when the first reception intensity becomes stronger than the threshold value, and generate the second instruction signal when a state in which the first reception intensity is stronger than the second reception intensity is changed to a state in which the second reception intensity is stronger than the first reception intensity.

6. The capsule endoscope system according to claim 3, wherein
the receiving device further includes a memory configured to chronologically store the image signal received by an antenna whose reception intensity is greatest among the first and the second antennas, and
when the transmitter wirelessly transmits at least one of the first and the second instruction signals, the controller is configured to generate a dummy image signal, insert the dummy image signal into a sequence of image signals stored in chronological order, and causes the memory to store the inserted dummy image signal.

7. The capsule endoscope system according to claim 3, wherein
the receiving device further includes a memory configured to chronologically store the image signal received by an antenna whose reception intensity is greatest among the first and the second antennas, and
when the transmitter wirelessly transmits at least one of the first and the second instruction signals, the controller is configured to add information to the image signal to be stored in the memory, the information indicating that the imaging frame rate is changed.

8. The capsule endoscope system according to claim 7, wherein the information is textual information or graphic information to be added to an image based on the image signal.

9. The capsule endoscope system according to claim 3, wherein the capsule endoscope further includes
a receiver configured to receive the first or the second instruction signal wirelessly transmitted from the receiving device, and
an imaging controller configured to change the imaging frame rate based on the first or the second instruction signal received by the receiver of the capsule endoscope.

10. A method for operating a capsule endoscope system comprising: a capsule endoscope configured to capture an image of inside of a subject at a changeable imaging frame rate, generate an image signal, and transmit a wireless signal including the image signal; and a receiving device including a first and a second antennas attached to a body surface of the subject at different positions and configured to receive the wireless signal transmitted from the capsule endoscope, the method comprising:
detecting, by the receiving device, a first reception intensity which is a reception intensity of the wireless signal at the first antenna and a second reception intensity which is a reception intensity of the wireless signal at the second antenna,
generating, by the receiving device, a first instruction signal that changes the imaging frame rate to a first value higher than an initial value that is set in advance when the first or the second reception intensity satisfies a predetermined condition, and
wirelessly transmitting, by the receiving device, the first instruction signal to the capsule endoscope.

11. The method for operating the capsule endoscope system according to claim 10, wherein the first instruction signal that changes the imaging frame rate to the first value higher than the initial value is generated when the first reception intensity becomes stronger than a threshold value.

12. The method for operating the capsule endoscope system according to claim 11, wherein
the generating includes generating a second instruction signal that changes the imaging frame rate to a second value lower than the first value when a strength relation of reception intensities between the first reception intensity and the second reception intensity is reversed, and
the transmitting includes transmitting the second instruction signal.

13. The method for operating the capsule endoscope system according to claim 12, wherein
the first antenna is attached to the body surface at the subject closer to an esophagus of the subject as compared with the second antenna,
the second antenna is attached to the body surface at the subject closer to a stomach of the subject as compared with the first antenna,
the first instruction signal is generated when the first reception intensity becomes stronger than the threshold value, and the second instruction signal is generated when a state in which the first reception intensity is stronger than the second reception intensity is changed to a state in which the second reception intensity is stronger than the first reception intensity, and
the transmitting includes transmitting the first and second instruction signals.
